(19) United States
(12) Patent Application Publication
(10) Pub. No.: US 2008/0188857 A1
(43) Pub. Date: Aug. 7, 2008

(54) Method and Device For Improving the Fixing of a Prosthesis

(76) Inventors: Lars Bruce, Viken (SE); Bengt Mjoberg, Ystad (SE); Lars Magnus Bjursten, Limhamn (SE)

Correspondence Address: SMITH, GAMBRELL & RUSSELL, 1130 CONNECTICUT AVENUE, N.W., SUITE 1130, WASHINGTON, DC, 20036, US

(21) Appl. No.: 11/663,272

(22) PCT Filed: Sep. 21, 2005
(86) PCT No.: PCT/SE05/01383

§ 371 Date: Nov. 9, 2007

Related U.S. Application Data
(60) Provisional application No. 60/611,390, filed on Sep. 21, 2004.

(30) Foreign Application Priority Data
Sep. 21, 2004 (SE) 0402276-0

(51) Int. Cl. A61B 17/58

(52) U.S. Cl. 606/94; 606/92; 606/93

(57) ABSTRACT

In a method and a device for improving the fixing of an elongate prosthesis in a bone cavity, said fixing being provided by a bed in which the prosthesis has been driven down by vibration, a ring of a flexible, essentially plastic material is arranged around the proximal part of the prosthesis so that the ring fills the space between the bone wall and the prosthesis, whereupon the ring is compacted and compressed, after which strokes are made on the prosthesis in its longitudinal direction for final fixing thereof in the bed. The ring significantly improves the strength of the prosthesis in the bed.

The invention relates to a method and a device for improving the fixing of a prosthesis according to the preamble to the independent claims.

The introduction of bone cement in the 1960s for fixing joint prostheses resulted in a dramatic improvement of the surgery results. Bone cement fills irregularities and takes care of insufficient fit between bone and prosthesis that arises due to varying anatomy or surgical inaccuracy. Bone cement thus provides an increased contact surface and a better and more evenly distributed load. However, bone cement also suffers from drawbacks. Thus, it comprises a toxic and allergenic monomer component, monomer methyl methacrylate, which leaks out from the cement composition and can enter the blood vessel during surgery, thus causing a fall in blood pressure in the patient, which requires cortisone treatment during the actual cementing. Bone cement hardens in the course of 10-15 min. sometimes at a relatively high temperature (due to, inter alia, the thickness of the cement), above 70° C. is not unusual, which may result in thermal damage to the bone. Moreover bone cement is brittle and there is a risk of fatigue fractures over time.

In the 1970s it was observed that cemented prostheses which initially seemed to be well fixed could sometimes come loose after several years. It was considered—and many still do consider—that such coming loose might be caused by cement particles. A large number of uncemented prostheses were marketed and expected to establish direct contact between bone tissue and prosthesis (osseointegration). Uncemented prosthesis fixation can be achieved by an accurate surgical technique and wedging of the prosthesis into the bone. However, this method has been found not perfectly reliable, and therefore the prosthesis has been provided with a porous surface or coated with hydroxyapatite in order to improve the possibility of bone ingrowth. The clinical results have varied from quite poor to good.

A serious problem in reoperation of loosened hip prostheses is the replacement of the bone that has been lost in connection with the loosening of the prosthesis. The common technique involves the use of pulverised bone. If possible, the patient's own bone is used, but in consideration of the fact that in most cases large amounts of material are required, frozen bone collected from other patients must be used. This involves a risk of transfer of infectious matter, such as HIV and hepatite virus. Various artificial bone substitutes have been tested over the years with different degrees of success. In reoperation, the prosthesis is fixed with cement in the bone-packed cavity. The clinical results have been good so far, but the intended bone ingrowth of the prostheses did not occur.

U.S. Pat. No. 5,015,256 discloses a different technique for fixing a prosthesis in a bone cavity. This technique does not use cement or packed pulverised bone but is instead based on the use of porous, irregular plastic grains consisting of biologically compatible material, having a size of preferably 0.5-2 mm, preferably of titanium, between the prosthesis (both for new operation and reoperation) and the inner wall of the bone cavity. The porous titanium grains induce bone ingrowth from the inner wall, through and between the grains and to the prosthesis and thus help to anchor the prosthesis in the bone cavity. A further advantage of the use of such grains is the elimination of the risk of infection. Moreover, they are not resorbed like packed pulverised bone. As a result, the conditions of maintaining the critical stability between bone and prosthesis will be significantly improved compared with natural bone as filling material.

According to U.S. Pat. No. 5,015,256, the reamed canal in the shaft of a femur is filled with titanium grains, and a femoral prosthesis is driven into the bed of titanium grains by a vibrating tool, a pneumatically driven, oscillating bone saw which strikes on the prosthesis head. In one embodiment, the initial vibration of the prosthesis stem occurs in an approximately horizontal circular arc back and forth at a first vibration frequency, thereby making the grains in the bed of grains fluidise, after which a motion occurs back and forth in the longitudinal direction of the stem at a second lower frequency, thereby packing the grains and locking them to each other. After vibration, a striking tool (hammer) is used to drive the prosthesis to its final locked position in the femur.

It has now been discovered that in application of this technique non-uniform compacting of the titanium grains in the femoral canal is obtained, where the compaction degree of the titanium grains varies along the prosthesis and is higher at the top than at the bottom during the entire compacting process and after it. This fact causes lower extraction resistance of the prosthesis than would be possible with a higher compaction degree at the top of the prosthesis. Moreover it has been discovered that the relatively low compaction degree at the top may cause ingrowth of connective tissue from the inner wall of the bone cavity to the prosthesis, which further impairs the fixing of the prosthesis at the top of the prosthesis since connective tissue is not capable of binding to the prosthesis, in contrast to bone tissue which grows from the inner wall of the bone cavity or decomposed biological material among the titanium grains.

A copending patent application, filed by the same applicant, discloses a technique of driving down by vibration a prosthesis into a body cavity filled with (titanium) grains (such as a femoral prosthesis into the femoral canal), which differs from the technique described above by the prosthesis being vibrated in a spiral motion oscillating back and forth, the axis of the vibrating tool essentially coinciding with the longitudinal axis of the prosthesis. This vibrating technique provides a distinctly more uniform compaction degree along the entire prosthesis and improved extraction resistance. However, it has been found that in connection with the final strokes on the prosthesis to drive it to the final position, the uppermost portion of the bed of grains is broken up, whereby part of the previously achieved uniformity in compaction along the prosthesis and the increased extraction resistance will be lost. Such breaking-up of grains, of course, also occurs in connection with the final strokes in the technique according to U.S. Pat. No. 5,015,256.

The object of the invention is, based on a technique of driving down a prosthesis into a body cavity essentially filled with biocompatible grains by vibration and final strokes, to eliminate or at least reduce the problems with non-uniform compacting and to improve the fixing of the prosthesis in the grain bed.

More specifically, the above object is achieved by a device according to claim 1, advantageous embodiments having the features stated in the dependent claims.

The device according to the invention prevents breaking up of the upper, that is distal, portion of the grain bed after driving down the prosthesis into the bed by vibration and during the final strokes, and provides a surface with high friction against the uppermost portion of the prosthesis stem, thereby considerably improving the extraction resistance, compared with the techniques described above. In addition, the swivelling resistance is improved (swivelling about the longitudinal axis of the prosthesis).

The invention will now be described in more detail with reference to the accompanying drawing, in which

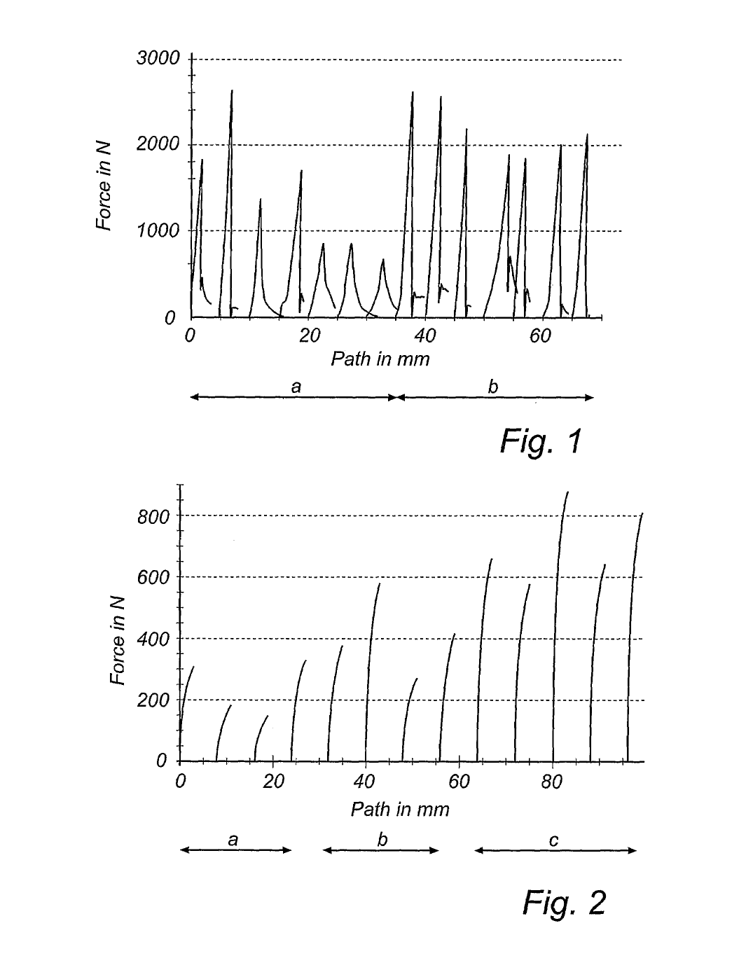

FIG. 1 shows comparative curves of extraction resistance and

FIG. 2 illustrates comparative curves of swivelling resistance.

As mentioned above, the invention is based on the technique of vibration and final strokes, which technique has been exemplified above and will therefore not be repeated in detail.

Based on the exemplification, the starting point of the invention is that the femoral prosthesis has been driven down by vibration into a grain bed in the femoral canal and it is time to strike on the prosthesis in its longitudinal direction for further driving down thereof into the grain bed and, thus, fixing thereof in the final position. The grain bed advantageously consists of about 0.5-2 mm porous, essentially plastic titanium grains, which reach about 1 cm below the trochanter. The prosthesis shoulder (the point of the prosthesis where the prosthesis stem and the prosthesis neck merge into one another) is positioned at the level of the trochanter and is to be driven down about 10-15 mm. Before driving down by strokes, the part of the prosthesis stem positioned above the grain bed is, according to the invention, enclosed by a substantially continuous formation, in the form of a ring, of a flexible, plastic or non-essentially elastic material. Moreover the ring is dimensioned so as to fill the ring space, free of grains, between said part of the prosthesis stem, below the prosthesis shoulder, and the inner wall of the bone canal enclosing this part. The ring is then compacted and compressed in the ring space downwards in the bed, which ring space tapers downwards in the bed, since both the femoral prosthesis and the femoral canal taper downwards, that is in the distal direction. Compacting and compressing can be performed, for instance, by a chisel, held by the surgeon's hand. In the ring space, a compacted and compressed ring is thus obtained, which fills the ring space and exerts pressure on the upper part of the prosthesis stem. The striking operation (one or more strokes) on the prosthesis is now performed. The strokes cause the prosthesis in its motion down into the bed to bring along the compressed and compacted ring, due to the frictional adhesion of the compressed and compacted ring to the prosthesis. Moreover the strokes cause additional compressing and compacting of the ring due to the conicity of the prosthesis stem. Such bringing along means that compressive force is exerted by the ring on the underlying grain bed, thereby preventing grains from being scattered about and slightly compressing and compacting the uppermost portion of the grain bed. Consequently the prosthesis acts as a piston rod and the compacted and compressed ring as a piston on the underlying bed, thus compressing it. If the outer surface of the prosthesis is rough or has projections, which it usually has, the bringing-along effect is increased. The surface of the compressed and compacted ring facing the prosthesis stem will exhibit strong frictional adhesion to the prosthesis stem.

Preferred Embodiment of the Ring

In a preferred embodiment, the ring is quite simply made of a thin sheet which is rolled, folded, wound, twisted, crumpled up or deformed in some other manner to a substantially elongate formation or string, which is bent as a ring with the ends of the formation facing each other or overlapping each other.

Thus the length of the formation should be such that the formation formed as a ring encloses the prosthesis a distance (for instance 1 cm) below the prosthesis shoulder, and the thickness of the formation should be such that the ring, enclosing the prosthesis in said place, is capable of filling the space between the prosthesis driven down by vibration and the inner wall of the bone canal up to the edge, in such a manner that it will be necessary to exert pressure on the ring to force it into said ring space to make it tightly fill the ring space in connection with pressing down. The outer diameter of the ring should thus be overdimensioned relative to the outer diameter of the ring space.

Pressing down can be performed using a handheld chisel or some other tool, with which it is possible to enter the ring space. It does not matter that the tool causes damage to the ring so as to deform it, damage the sheet material, on the contrary it is advantageous since the contact surface between ring and prosthesis can thus be increased. The main thing is that the pressing down operation results in a pressed-down ring—different from the ring as started with—on the free distal surface of the bed, which in a tight, compacted and compressed manner fills the ring space between prosthesis and femoral canal.

Ring Alternative

Completed rings (that is manufactured endless) can be used and are within the scope of the invention. However, they have the drawback that differently dimensioned rings must be provided to fit femoral canals of different widths. The preferred sheet of a suitable length makes it possible (for the surgeon) to provide any suitable formation as described above.

However, it will be appreciated that to ensure a ring which is pressed down in the ring space as described above and which can function as a pressure piston, it is not necessary to produce a ring of an elongate formation in advance. It is perfectly possible to produce the ring in the actual ring space by placing an end of the formation in the ring space and then gradually placing the rest of the formation in the ring space until a ring is formed.

In the event of failure to fill the space with a pressed-down ring, for instance by using too small an amount of material to provide a filling ring, it is possible to produce a complementary ring or fill the gap between the ends of an arch with extra material of the same kind as used in the non-filling ring or arch.

The ring can also be made of a “waste wool”-like structure or “steel wool”-like structure.

The Preferred Sheet

The sheet of plastic or non-essentially elastic material is advantageously perforated so that bone tissue from decomposed biological material in the granular mass or from the bone canal wall can penetrate through and up to the prosthesis, which increases the fixing of the prosthesis in the grain bed. Preferably, the sheet is a net, for instance knitted or woven, or punched by, for example, laser punching. However, the sheet can also be compact, that is unperforated.

Ring Material

As material for the ring a plastic or non-essentially elastic material has been mentioned above. The material is required to be plastic since otherwise its elastic pressure would damage the wall of the bone canal. Precisely for the same reason, the grains according to U.S. Pat. No. 5,015,246 are also of a material which is plastic or non-essentially elastic.

As material for the ring, the same material as for the grains is preferred, especially titanium owing to its bioreactivity, which allows bone tissue to bind thereto. Another suitable bioreactive material is tantalum. Different materials for the grains and the ring material should be avoided to prevent generation of electric currents. However the ring material can be some other biocompatible material, such as plastic.

### EXAMPLE

Extraction tests have been performed with the device according to the invention inserted into a grain bed with and without a fixing ring of titanium net, FIG. 1. This Figure shows a distinctly improved extraction resistance of a prosthesis with a fixing ring according to the invention; “a” is the result without a titanium net and “b” is the result with a titanium net.

Moreover turning tests have been performed with the device according to the invention, with and without a fixing ring, and also without a fixing ring but with the granular mass at the top of the bone canal compacted in points around the prosthesis, FIG. 2. FIG. 2 demonstrates that point-by-point compaction of granular mass directly, that is not via a ring according to the invention, is not efficient for optimal fixing. “a” is the result without a titanium net and without compacting, “b” is the result without a titanium net and with point-by-point compacting and “c” is the result with a titanium net.

In the above-mentioned tests, the grains consisted of 0.5-2 mm essentially plastic or essentially non-elastic titanium grains irregular in shape. The prosthesis was made by CLS.

The bone cement cover disclosed in U.S. Pat. No. 5,015,256 is not necessary since it is replaced by the ring.

A femoral prosthesis has been described above as an example of the application of the invention. It will be appreciated, however, that the invention is applicable to other elongate prostheses for insertion into corresponding elongate body cavities, such finger prostheses, arm prostheses, dental prostheses.

1. A device for improving the fixing of an elongate prosthesis in a body cavity, said fixing being performed by a bed of biocompatible granular material, in which bed the prosthesis has been driven down by vibration and in which bed the prosthesis has been finally fixed by striking on the prosthesis in its longitudinal direction, the prosthesis having a part positioned in the body cavity and projecting from the bed of grains, characterised in that it comprises a substantially continuous ring of a flexible, plastic or non-essentially elastic material, which is compacted and compressed and fills out the distance between said prosthesis part projecting from the bed and the opposite wall of the body cavity.

2. A device as claimed in claim 1, characterised in that the ring is made of a sheet, which is deformed to an elongate formation and then bent as a ring.

3. A device as claimed in claim 2, characterised in that the sheet material is a net.

4. A device as claimed in any one of claims 1-3, characterised in that the ring material is biocompatible metal.

5. A device as claimed in any one of claims 1-4, characterised in that the ring material is titanium.

6. A device as claimed in any one of claims 1-5, characterised in that the ring material is the same as the granular material.

7. A method for improving the fixing of an elongate prosthesis in a body cavity, said fixing being performed by a bed of biocompatible granular material, in which bed the prosthesis has been driven down by vibration and in which bed the prosthesis has been finally fixed by striking on the prosthesis in its longitudinal direction, characterised by arranging, after said driving down by vibration but before said final fixing by striking, a flexible, plastic or non-essentially elastic material in a ring space, essentially free of grains, around the proximal part of the prosthesis, so that the ring encloses this part and fills the ring space, compacting and compressing the ring in the ring space during or after said arranging, and subsequently performing the final fixing by strokes.

8. A method as claimed in claim 6, characterised by making the ring of a sheet, which is deformed to an elongate formation and is then bent as a ring.

9. A method as claimed in claim 7 or 8, characterised in that the sheet is a net.

10. A method as claimed in any one of claims 7-9, characterised in that the ring material is titanium.

11. A method as claimed in any one of claims 7-10, characterised by forming the ring while being arranged in said ring space.

12. A method as claimed in any one of claims 7-10, characterised by forming the ring in advance.
